(19) United States
(12) Patent Application Publication
(10) Pub. No.: US 2008/0181972 A1
(43) Pub. Date: Jul. 31, 2008

(54) Compositions and Methods for Maintaining, Strengthening, Improving, or Promoting Eye Health

(76) Inventors: Valentina Amico, Tremestieri (IT); Stephen P. Bartels, Pittsford, NY (US); Karl Cummins, Hilton, NY (US); Daniel Stein, Rochester, NY (US)

Correspondence Address: Bausch & Lomb Incorporated, One Bausch & Lomb Place, Rochester, NY, 14604-2701, US

(21) Appl. No.: 11/969,239

(22) Filed: Jan. 4, 2008

Related U.S. Application Data
(60) Provisional application No. 60/886,956, filed on Jan. 29, 2007.

(51) Int. Cl. A61K 33/24; A61K 31/385; A61K 31/51; A61P 3/00; A61P 27/02

(52) U.S. Cl. 424/655; 514/440; 514/276

(57) ABSTRACT

Compositions for maintaining, strengthening, improving, or promoting eye health comprise α-lipoic acid, ascorbic acid, and α-tocopherol, or pharmaceutical equivalents thereof. The compositions are useful to prevent, stabilize, reverse and/or treat visual acuity loss in subjects having, or being at risk of developing, an ocular disease. The compositions can further comprise one or more other antioxidants, beneficial minerals, and/or nutritional or dietary materials.

### CROSS-REFERENCE

This application claims the benefit of Provisional Patent Application No. 60/886,956 filed Jan. 29, 2007, which is incorporated by reference herein.

### BACKGROUND OF THE INVENTION

The present invention relates to compositions and methods for maintaining, strengthening, improving, or promoting eye health. In particular, the present invention relates to nutritional or dietary supplement compositions and methods for maintaining, strengthening, improving, or promoting eye health in people with particular ocular diseases. More particularly, the present invention relates to compositions and methods for maintaining, strengthening, improving, or promoting eye health in patients having or being at risk to develop diabetic ocular complications or ocular inflammation.

Diabetes is a chronic illness that requires continual medical care and patient self-management education to prevent acute complications and to reduce the risk of long-term complications. Diabetes mellitus can result from a variety of genetic, metabolic, and acquired conditions eventuating in hyperglycemia. The pathology of diabetes is characterized by metabolic derangements in the metabolism of glucose and abnormalities in metabolism of fat, protein and other substances. All forms of diabetes are characterized by chronic hyperglycemia, attributable to either insulin insufficiency or insulin resistance and the development of diabetes-specific microvascular pathology in the retina, renal glomerulus and peripheral nerve. As a consequence of its microvascular pathology, diabetes is a leading cause of blindness, end-stage renal disease and a variety of debilitating neuropathies. Diabetes is also associated with accelerated atherosclerotic macrovascular disease affecting arteries that support the heart, brain and lower extremities. As a result, patients with diabetes have a much higher risk of myocardial infarction, stroke and limb amputation. M. Brownlee, Nature, Vol. 414, 813 (2001).

Diabetic Retinopathy (“DR”) is a highly specific vascular complication of the eye of patients having either type-1 or type-2 diabetes. The prevalence of retinopathy is strongly related to the duration of diabetes. The risk of retinopathy is directly related to the degree and duration of hyperglycemia. American Diabetes Association, Diabetes Care, Vol. 28, Supp. 1, S4 (2005).

DR is estimated to be the most frequent cause of new cases of blindness among adult aged 20-74 years. American Diabetes Association, Diabetes Care, Vol. 28, Supp 1, S4 (2005). The risk of retinopathy is directly related to the degree and duration of hyperglycemia. After diabetes mellitus has been present for 20 years, almost all persons in whom the onset of diabetes occurred before the age of 30 years have some evidence of retinopathy, and about half have proliferative retinopathy. Persons who are 30 years or older when diabetes develop are at lower risk for retinopathy, but in this group retinopathy may be the first sign of diabetes. F. Ferris et al., Drug Therapy, Vol. 341, 667 (1999). The earliest clinical signs of DR are vascular lesions including microaneurysms, small outpouchings from retinal capillaries, dot intraretinal hemorrhages and vasodilatation. As the disease progresses, patients with proliferative diabetic retinopathy (“PDR”) have an increase in the number and size of intraretinal hemorrhages and new blood vessels develop from the retinal circulation. These new vessels can extend into the vitreous cavity of the eye and can hemorrhage into the vitreous, resulting in visual loss, and they can cause tractional retinal detachments from the accompanying contractile fibrous tissue. Late in the course of the disease, new blood vessels may form within the stroma of the iris and may extend, with accompanying fibrosis, into the structures that drain the anterior chamber angle of the eye. This development blocks the outflow of aqueous humor, causing neovascular glaucoma, with a devastating elevation of the intraocular pressure. R. N. Frank, New Engl. J. Med., Vol. 350, 48 (2004).

Chronic hyperglycaemia commonly found in people with diabetes can lead to enhanced levels of reactive oxygen species within cells. While oxidation is a normal part of cell metabolism, an imbalance between enhanced production of reactive oxygen species and insufficient capacity of the cellular antioxidant defense systems can result in oxidative stress. This oxidative stress can result in activation of a number of cellular pathways that may contribute to DR such as polyol, hexosamine, protein kinase C and advanced glycation and lipoxidation endproduct (“AGE/ALE”) formation.

The polyol pathway becomes active when intracellular glucose levels are elevated. Its activation may result in biochemical changes leading to altered intracellular metabolism causing “cell swelling” and exacerbated oxidative stress. J. H. Kinoshita, Invest. Opthalmol. Vol. 13, 713 (1974); Van den Enden et al., IOVS Vol. 36, 1675 (1995); M. Brownlee, Nature, Vol. 414, 813 (2001).

Activation of the hexosamine pathway by hyperglycaemia may result in changes in both gene expression and protein function, which together contribute to the pathogenesis of some diabetic complications mainly hyperlipidaemia, obesity and impaired glucose tolerance. Rumberg et al., J. Biol. Chem., Vol. 278, 28547 (2003); Verababu et al., Diabetes, Vol. 49, 2070 (2000).

Hyperglycaemia is considered responsible for changes in the microcirculation including microvascular endothelial dysfunction and capillary leakage which may represent the initiating mechanisms that underlie the pathogenesis of microangiopathic complications (circulatory disorders such as cardiomyopathy, angiopathy and atherosclerosis). The abnormal activation of protein kinase C (“PKC”) is reported to be responsible for many retinal capillary dysfunctions and lesions such as micro-aneurysms, increases in vascular permeability, basement membrane thickening, alteration of retinal blood flow and neovascularization. Way et al., Diabet. Med. Vol. 18, 945 (2001).

Formation of AGE/ALE is one of the underlying factors contributory to the development of complications of diabetes. Accumulation of AGE/ALE, resulting in the carbonyl stress, has been linked with diabetic vascular complications and increased oxidative stress. M. Brownlee, Nature, Vol. 414, 813 (2001); Yan et al., J. Biol. Chem. Vol. 269, 9889 (1994); A. W. Stitt, Br. J. Opthalmol. Vol. 85, 764 (2001).

It has been recently reported that a common element links the above mentioned pathogenic mechanisms. Each of them, in fact, reflects a single hyperglycaemia-induced process: overproduction of superoxide by the mitochondrial electron-transport chain. Brownlee M, Nature, Vol. 414, 813 (2001). The increase in glycoxidation and lipoxidation products in plasma and tissue proteins suggests that oxidative stress is increased in diabetes and it can be considered a cause and a consequence of the pathogenic mechanisms leading to the diabetic complications. Several mechanisms, including autooxidative glycation, formation of AGE/ALE and increased polyol pathway activity contribute to increasing oxidative stress, but these mechanisms overlap and intersect with one another. For example, AGE formation and altered polyol pathway activity may lead to oxidative stress, oxidative stress may accelerate AGE formation, and reductive stress may lead to activation of PKC, and so on. J. W. Baynes et al., Diabetes, Vol. 48, 1 (1999); P. S. Van Dam, Diabetes Metab. Res. Rev., Vol. 18, 176 (2002).

Diabetes results in an imbalance between the production of free radical species and the defense against them. The term free radical species includes ROS: superoxide anion (O₂⁻), hydrogen peroxide (H₂O₂), hydroxyl radical (HO⁺) and reactive nitrogen species such as nitric oxide (NO). O₂⁻ is of particular interest because it can react with NO producing the high reactive peroxynitrite (ONOO⁻), which can result in cytotoxicity due to lipid peroxidation, inactivation of enzymes by oxidation of protein sulphydryls and nitration of tyrosines and damage to DNA and mitochondria. Therefore, increased oxidative stress, which contributes to the pathogenesis of diabetic complications, is the consequence of either enhanced free radicals production or attenuated free radical scavenging capacity. Oxidative and nitrosative stress contribute to the vascular endothelial cell damage by causing breakdown of the blood-retinal barrier that characterizes the early stages of vascular dysfunction in diabetes. Y. Du et al., Free Rad. Biol. Med., Vol. 35, 1491 (2003); A. B. El-Remessy et al., Am. J. Pathol., Vol. 162, 1995 (2003).

Laser photocoagulation is the current mainstay of therapy for DR and it is indicated essentially for all patients when retinopathy progresses to the more advanced proliferative stages. Unfortunately, this therapy is associated with side effects such as a decrease in peripheral and night vision and changes in color perception. Moreover, in some instances, retinopathy continues to progress despite timely and appropriate laser photocoagulation. Under rare potentially serious circumstances, complications of laser therapy may also occur. L. P. Aiello, Surv. Opthalmol., Vol. 47, S263 (2002).

Therefore, there is a continued need to provide improved therapeutic methods for treating, stabilizing, or reversing diabetic ocular complications. It is also very desirable to provide such methods in a non-invasive manner. Moreover, it is very desirable to provide such methods through simple nutritional or dietary supplements.

### SUMMARY OF THE INVENTION

In general, the present invention provides a nutritional or dietary supplement composition for administration to humans or other animals that maintains, strengthens, improves, or promotes ocular health thereof.

In one aspect, said nutritional or dietary supplement composition maintains, strengthens, improves, or promotes ocular health of patients having, or being at risk to develop, ocular diseases.

In another aspect, administration of a composition of the present invention can prevent, stabilize, reverse and/or treat visual acuity loss in patients with ocular diseases.

In another aspect, administration of a composition of the present invention can prevent, stabilize, reverse and/or treat visual acuity loss in patients with diabetic ocular complications.

In still another aspect, a nutritional or dietary supplement composition of the present invention can also be administered to prevent, stabilize, reverse and/or treat complications of diabetic retinopathy, diabetic macular edema, cataract, glaucoma, or ocular inflammation (such as uveitis).

In still another aspect, a nutritional or dietary supplement composition of the present invention comprises amounts of specific antioxidants effective to maintain, strengthen, improve, or promote ocular health of patients having, or being at risk to develop, ocular diseases.

In still another aspect, a nutritional or dietary supplement composition of the present invention comprises amounts of specific antioxidants effective to prevent, stabilize, reverse and/or treat complications of diabetic retinopathy, diabetic macular edema, cataract, glaucoma, or ocular inflammation.

In yet another aspect, a nutritional or dietary supplement composition of the present invention comprises α-lipoic acid, ascorbic acid, and α-tocopherol.

In yet another aspect, a nutritional or dietary supplement composition of the present invention comprises α-lipoic acid, genistein, ascorbic acid, and α-tocopherol.

In a further aspect, a nutritional or dietary supplement composition of the present invention can decrease visual acuity loss in diabetic patients.

The present invention also provides a method of maintaining, strengthening, improving, or promoting ocular health of a patient having, or being at risk to develop, ocular diseases. The method comprises administering to said patient a nutritional or dietary supplement composition that comprises effective amounts of specific antioxidants to strengthen, improve, or promote ocular health of said patient.

In still another aspect, the practice of this invention involves supplementing the diet of humans or animals by oral, intraperitoneal, intravenous, subcutaneous, transcutaneous, and/or intramuscular routes of administration with said antioxidants.

In yet another aspect, the present invention also provides a method of manufacturing a nutritional or dietary supplement composition. The method comprises combining specific antioxidants in desired amounts, in a dosage form.

Other features and advantages of the present invention will become apparent from the following detailed description and claims.

### DETAILED DESCRIPTION

In general, the present invention provides a nutritional or dietary supplement composition for administration to humans or other animals that strengthens, improves, or promotes ocular health of patients suffering from, or being at risk to develop, ocular diseases.

In one aspect, such ocular diseases include diabetic ocular diseases, cataract, glaucoma, ocular inflammation, and combinations thereof.

In one aspect, administration of a composition of the present invention can prevent, stabilize, reverse, and/or treat visual acuity loss in patients with diabetic ocular complications or ocular inflammation.

In another aspect, a nutritional or dietary supplement composition of the present invention comprises α-lipoic acid, ascorbic acid, and α-tocopherol.

In yet another aspect, a nutritional or dietary supplement composition of the present invention comprises α-lipoic acid, genistein, ascorbic acid, and α-tocopherol.

In still another aspect, a nutritional or dietary supplement composition of the present invention comprises the ingredients disclosed in Table 1, each present in amounts shown therein.

TABLE 1

| Ingredient | Daily Dosage |
|---|---|
| α-lipoic acid | 50-1000 mg |
| ascorbic acid | 50-1000 mg |
| α-tocopherol | 10-1000 mg |

In still another aspect, a nutritional or dietary supplement composition of the present invention comprises the ingredients disclosed in Table 2, each present in amounts shown therein.

TABLE 2

| Ingredient | Daily Dosage | |
|---|---|---|
| α-lipoic acid | 50-1000 | mg |
| genistein | 10-200 | mg |
| ascorbic acid | 50-1000 | mg |
| α-tocopherol | 10-1000 | mg |

In one aspect, each of the ingredients listed in Table 1 or 2 can be replaced by one of its derivatives that are therapeutically or nutritionally equivalent to the ingredient. A therapeutically or nutritionally equivalent compound to an ingredient is also referred to herein sometimes as a “pharmaceutically equivalent” compound and means a compound that can produce the same therapeutic or nutritional effect in a subject as the ingredient in question. A pharmaceutically equivalent compound is herein also called a “pharmaceutical equivalent.” However, it should be recognized by a person skilled in the art that a pharmaceutically equivalent compound may be included in a composition in a different amount in order to produce the same level of effect as the ingredient in question. For example, each of α-lipoic acid, ascorbic acid, and α-tocopherol can be replaced by one of its pharmaceutically acceptable salts or esters in therapeutically or nutritionally equivalent amounts. However, it should be noticed that, as used herein, a pharmaceutical equivalent to an ingredient can be a derivative thereof or another compound that is substantially different in chemical structure but provides substantially equivalent therapeutic or nutritional effect to the ingredient in question. In one embodiment, a pharmaceutical equivalent to an ingredient is a derivative thereof that provides the same therapeutic or nutritional effect as the ingredient.

In another aspect, a composition of Table 1 or 2 further comprises one or more other antioxidants and/or one or more essential nutrients or minerals.

In still another aspect, when an ingredient can exist in different isomeric forms, it can be included in a composition of the present invention in any isomeric form or as a mixture of isomers.

In a further aspect, the amount of α-lipoic acid in a daily dosage of a composition of the present invention is in the range of 100-600 mg. Alternatively, such amount is in the range of 100-400 mg, or 100-300 mg, or 200-600 mg, or 200-400 mg, or 300-400 mg, or 300-600 mg. When a therapeutically or nutritionally equivalent derivative of α-lipoic acid is used in a daily dosage of a composition of the present invention, the amount of such equivalent is in a range that can produce a nutritional or therapeutic effect of an amount of α-lipoic acid in one of the ranges disclosed above.

In still another aspect, the amount of genistein in a daily dosage of a composition of the present invention is in the range of 10-150 mg. Alternatively, such amount is in the range of 20-100 mg, or 20-80 mg, or 50-100 mg, or 50-80 mg, or 50-70 mg, or 10-50 mg, or 10-40 mg, or 10-30 mg. When a therapeutically or nutritionally equivalent derivative of genistein is used in a daily dosage of a composition of the present invention, the amount of such equivalent is in a range that can produce a nutritional or therapeutic effect of an amount of genistein in one of the ranges disclosed above.

In a further aspect, the amount of ascorbic acid in a daily dosage of a composition of the present invention is in the range of 100-600 mg. Alternatively, such amount is in the range of 100-400 mg, or 100-300 mg, or 200-600 mg, or 200-400 mg, or 200-300 mg, or 300-600 mg. When a therapeutically or nutritionally equivalent derivative of ascorbic acid is used in a daily dosage of a composition of the present invention, the amount of such equivalent is in a range that can produce a nutritional or therapeutic effect of an amount of ascorbic acid in one of the ranges disclosed above.

In a further aspect, the amount of α-tocopherol in a daily dosage of a composition of the present invention is in the range of 10-600 mg. Alternatively, such amount is in the range of 10-50 mg, or 10-100 mg, or 10400 mg, or 20-600 mg, or 20-400 mg, or 20-300 mg, or 100-600 mg, or 100400 mg, or 200-600 mg, or 200-400 mg, or 200-300 mg, or 300-600 mg, or 300-400 mg. When a therapeutically or nutritionally equivalent derivative of α-tocopherol is used in a daily dosage of a composition of the present invention, the amount of such equivalent is in a range that can produce a nutritional or therapeutic effect of an amount of α-tocopherol in one of the ranges disclosed above.

In one aspect, a composition of the present invention comprises a range of different antioxidants that can provide oxidative stress relief on ocular tissues at risk of being damaged or progressing toward a significant pathological condition, as a result of complications of diabetes. Some of the antioxidants of the present composition can act synergistically or on different physiological targets to provide enhanced benefits to the patients.

In another aspect, a composition of the present invention further comprises an additional material that confers a health benefit.

In another aspect, such an additional material reduces an adverse condition of a diabetic patient. In one embodiment, such adverse condition is a diabetic ocular complication. In another embodiment, such a diabetic ocular complication includes a retinal complication of diabetes. In another embodiment, such a diabetic ocular complication comprises diabetic retinopathy, diabetic macular edema, or both.

In still another aspect, such an additional material comprises vitamin B₁, vitamin B₃, vitamin B₆, essential minerals, derivatives thereof, and combinations thereof.

In yet another aspect, such an additional material is present in a composition of the present invention in an amount in the range from about 100% to about 300% of the U.S. recommended dietary allowance (“RDA”), or alternatively, from about 100% to 200% of the RDA, for the age group and gender of the patient.

In a further aspect, the essential mineral comprises chromium(III). In one embodiment, chromium(III) is present in a composition of the present invention in the form of a pharmaceutically acceptable compound. In certain embodiments, the amount of chromium(III) in a composition is in the range from about 0.05 mg to about 0.5 mg (measured as chromium).

A composition of the present invention can be formulated in the form of tablets, caplets, capsules, gels, syrups, solutions, dispersions, emulsions, patches, or the like. Other forms also are possible, depending on the mode of administration. In one embodiment, a composition of the present invention is formulated for oral administration into a patient.

In one embodiment, a daily dosage of a composition of the present invention, each ingredient of which is in a range specified herein, may be administered one, two, three, four, or more times per day. Preferably, a daily dosage of a composition of the present invention is provided in the form of one tablet taken twice daily, for a total of two tablets a day, or in the form of two tablets taken twice daily, for a total of four tablets a day. Compared to taking the total daily dose once a day, twice daily dosing of half the total daily dose in one or more tablets per dose provides improved absorption and better maintenance of blood levels of the essential ingredients.

Since ingredients are subject to degradation over time, a composition preferably contains quantities of ingredients larger than the minimum disclosed herein to compensate for ingredient degradation. For example, the quantity of each ingredient is provided in a composition such that a minimum desired quantity is ensured through the expiration date of the composition on the sale label. The rate of degradation of each ingredient can depend on its sources, which should be taken into account in formulating the composition at the time of manufacture. Thus, the specific formulation of a composition can vary depending on the sources of the individual ingredients and the specified length of product shelf life before expiration. Typically, the product shelf life for nutritional or dietary supplements is approximately two to three years. Formulations may also vary somewhat depending on slight deviations from manufacturing specifications within controlled tolerance ranges as customary within the field of art. However, a composition comprising the minimum quantity of each ingredient disclosed herein can still find nutritional or dietary utility.

Variations contemplated in administering the subject composition to humans or other animals include, but are not limited to, providing time-release tablets or tablets manufactured to be administered as a single dose or as other multiple part dosages. Additionally, alternative avenues of administration besides oral administration are contemplated herein such as for example, but not limited to, intraperitoneal, intravenous, subcutaneous, sublingual, transcutaneous, intramuscular, or like forms of administration.

### Alpha-Lipoic Acid

Alpha-lipoic acid (“ALA”) provides superior antioxidant protection because, in addition to its own antioxidant activity, it is able to regenerate other antioxidants in the body. ALA is a low molecular weight substance which can be synthesized by both animals and humans. ALA may be considered a “metabolic antioxidant” because it is intimately connected to cell metabolism and redox state. First, ALA is an essential co-factor in mitochondrial α-ketoacid dehydrogenase complexes; hence it is essential for normal oxidative metabolism. Second, the reduction of ALA into dihydrolipoic acid (“DHLA”), that involves both NADH and NADPH, may modulate NADH/NAD⁺ and NADPH/NADP⁺ ratios, thus affecting numerous aspects of the cell metabolism. L. Packer et al., Free Rad. Biol. Med., Vol. 22, 359 (1997). ALA, and its reduced form DHLA, meet all the criteria making ALA an “ideal antioxidant”. It has been called “a universal antioxidant.” V. E. Kagan et al., Biochem. Pharmacol., Vol. 44, 1637 (1992).

### Amphiphilic Character

ALA shows its antioxidant effect in both fat- and water-soluble media. Furthermore, its antioxidant activity extends to both the oxidized form and its reduced form (DHLA). Exogenously supplied ALA is readily absorbed from the diet, transported, taken up by cells, and rapidly converted to DHLA in various tissues. The DHLA thus formed is also exported from the cells and can provide antioxidant protection to the extracellular compartment and to nearby cells; hence protection is afforded to both intracellular and extracellular environments. L. Packer et al., Free Rad. Biol. Med., Vol. 19, 227 (1995). DHLA in contrast to many other antioxidants may function as a “universal free radical quencher” who can scavenge peroxyl radicals both in the cytosol and in the hydrophobic membrane domains. V. E. Kagan, Biochem. Pharmacol., Vol. 44, 1637 (1992).

### Radical Quenching Activity

ALA scavenges hydroxyl radicals, hypochlorous acid, NO, ONOO—, H₂O₂, and singlet oxygen. DHLA is an even more potent antioxidant; in addition, it scavenges superoxide radicals and peroxyl radicals actively formed in many metabolic processes and during the course of lipid peroxidation. L. Packer et al., Free Rad. Biol. Med., Vol. 22, 359 (1997).

### Metal Chelation Activity

Metal chelation is a property of a compound that can result either in antioxidant or in pro-oxidant activity. Antioxidant activity is obtained when the electron density is isolated from metal to chelator, so electrons cannot be transferred to O₂. ALA/DHLA show antioxidant activity through chelation vs. transition metals, such as iron, copper, cadmium which can induce free radical damage in biological systems by catalyzing the decomposition of hydroperoxides, thus generating highly toxic hydroxyl radicals. G. P. Biewenga et al., Gen. Pharmac., Vol. 29, 315 (1997).

Capacity to Interact with Other Antioxidants

DHLA appears to be able to regenerate other antioxidants. It is a strong reductant regenerating oxidized antioxidants such as ascorbate, glutathione, or coenzyme Q, all of which can contribute to Vitamin E regeneration from its radical oxidized form, as well as reducing thioredoxin. Vitamin E is the major chainbreaking antioxidant that protects membranes from lipid peroxidation. When Vitamin E scavenges a peroxyl radical, a Vitamin E radical is formed. The Vitamin E radical may be reduced at the interface between lipid and water by several antioxidants such as ascorbate, ubiquinol and reduced glutathione (“GSH”). DHLA can reduce all these antioxidants and be regenerated by several enzymes, including lipoamide reductase, GSH reductase and thioredoxin reductase. Therefore, ALA and DHLA take central position in the antioxidant network. L. Packer et al., Nutrition, Vol. 17, 888 (2001). Vitamin E exists in biological membranes in a low molar ratio to unsaturated phospholipids (usually less than 0.1 nmol per mg of membrane protein: one molecule per 1000 to 2000 membrane phospholipid molecules, which are the main targets of oxidation in membranes). Lipid peroxyl radicals can be generated in membranes at the ratio of 1-5 nmol per mg of membrane protein per minute. Nevertheless, destructive oxidation of membrane lipids does not normally occur nor is Vitamin E rapidly depleted. These apparent paradoxes can be explained by “Vitamin E recycling” in which the antioxidant ability of Vitamin E is continuously restored by other antioxidants. L. Packer et al., Free Rad. Biol. Med., Vol. 19, 227 (1995).

Moreover, ALA can cause an increase in intracellular GSH which is essential in protecting neurons from excitotoxic insult. It has been shown that ALA administration increases intracellular GSH levels by 30-70%, both in vitro (cell culture) and in vivo (in lung, liver and kidney cells of mice injected daily with doses of 4, 8, or 16 mg/kg of ALA for 11 days). Such elevation in GSH cannot be explained by reduction of oxidized (disulphide) glutathione (“GSSG”), since GSSG is normally present at less than 10% the concentration of GSH. E. Busse et al., Neurotherapeutika Neurotherapeutic Drugs, Vol. 6, 829 (1992). Thus, it appears that ALA/DHLA act as antioxidants not only directly, through radical quenching and metal chelation, but indirectly as well, through recycling of other antioxidants and possibly through the induction of increased intracellular levels of GSH. L. Packer et al., Free Rad. Biol. Med., Vol. 22, 359 (1997).

### Genistein

Genistein is a phytoestrogen with a wide variety of pharmacological effects. Dietary genistein has been linked, through epidemiological and animal model studies, with a range of potential health beneficial effects (chemoprevention of breast and prostate cancer, cardiovascular disease and post-menopausal ailments). Genistein is synthesized in plants from the flavonone naringenin by a reaction catalysed by the cytochrome P450 enzyme isoflavone synthase (“IFS”). IFS genes have been cloned from a number of plant species and production of genistein can be now achieved in non-legumes by recombinant DNA approach. R. A. Dixon, et al. Phytochemistry, Vol. 60, 205 (2002).

### Effects on Oxidative Stress

Soy isoflavones have shown antioxidant properties in multiple studies utilizing in vitro assays.

### Radical Quenching Activity

Isoflavones have direct free radical quenching ability, with genistein and daidzein being particularly effective. Wei et al. reported that genistein strongly inhibited tumor promoter-induced H₂O₂ formation, both in vitro and in vivo, by suppressing H₂O₂ production. Wei, Nutr. Canc., Vol. 20, 1 (1993). Ruiz-Larrea et al. determined the antioxidant activities of a range of phytoestrogenic isoflavones. The results showed that the order of reactivity in scavenging the radical in the aqueous phase was genistein>daidzein=genistin approximately equal to biochanin A=daidzein>formononentin approximately equal to onionin. Furthermore, the examination of the abilities of these compounds to enhance the resistance of LDL to oxidation supported the observation that genistein was one of the most potent antioxidants among this family of compounds studied. M. B. Ruiz-Larrea, Free Rad. Res., Vol. 26, 63 (1997). Experiments by Zielonka et al. with pulse radiolysis showed that genistein possessed both peroxy- and hydroxyl-radical scavenging properties. J. Zielonka et al., Free Radic. Biol. Med., Vol. 35, 958 (2003). Kurk et al. reported the results of a complex study of antioxidant activity of genistein and oleuropein (a plant-derived phenolic compound), using electron spin resonance, chemiluminescence, fluorescence and spectrophotometric techniques. Their results confirmed good scavenging activity towards O₂⁻, HO⁺ and ROO⁺ as well as the antioxidant effect of genistein and oleuropein. I. Kruk et al., Luminescence, Vol. 20, 81 (2005).

### Antioxidant Effects on LDL

The effect of isoflavones on susceptibility of LDL to oxidative modification has been also investigated. Safari et al. evaluated the ability of some flavonoids to reduce LDL oxidizability estimated by measuring conjugated diene, lipid peroxides and TBARS formation after cupric sulfate solution was added. The following order was found: quercetine>morin>pelargonidin>genistein>naringin>apigenin. M. R. Safari et al., Prostaglandins Leukot. Essent. Fatty Acids, Vol. 69, 73 (2003). Particularly, Kapiotis et al. investigated the ability of genistein to act as an LDL antioxidant and protective agent against oxidized LDL (oxLDL) damage to vascular endothelial cells. In presence of genistein, bovine aortic endothelial cell- and human endothelial cell-mediated LDL oxidation was inhibited. S. Kapiotis, Arterioscler. Thromb. Vasc. Biol., Vol. 17, 2868 (1997). Exner et al. showed a protective effect of genistein on LDL atherogenic modification at glucose/genistein molar ratio which may occur in vivo suggesting a beneficial action of a soy diet in preventing chronic vascular diseases. M. Exner et al., Free Rad. Res., Vol. 34, 101 (2001).

### Ascorbic Acid

Ascorbic acid or vitamin C is a well-known water-soluble antioxidant. It provides in vivo antioxidant protection primarily as an aqueous phase peroxyl and oxygen radical scavenger. R. A. Jacob and B. J. Burri, Am. J. Clin. Nutr. Vol. 63, 985S (1996). Its activity is expressed through a variety of mechanisms. In principal it acts by scavenging ROS directly. Moreover, although ascorbic acid cannot scavenge lipophilic radicals within the lipid compartment by itself, it acts as a synergist with tocopherol for the reduction of lipid peroxyl radicals within the lipid compartment by reacting with tocoperoxyl radical and regenerating active tocopherol. E. Niki, Am. J. Clin. Nutr. Vol. 54, 1119S, (1991). Humans depend on external sources of vitamin C to meet their vitamin C requirements. Vitamin C in the form of ascorbate is found in the aqueous humor of human eyes. A high concentration of ascorbate in the aqueous humor of eyes is maintained by active transport of ascorbate from the blood stream to the posterior chamber of the eyes. Maximum aqueous humor ascorbate concentration occurs with a blood plasma ascorbate level in the range of approximately 0.3 to 0.5 milligram/deciliter (mg/dl).

The U.S. recommended dietary allowance (RDA) for vitamin C in the form of ascorbic acid is 60 mg. Very large daily doses of vitamin C have been taken over many years with no or only minor undesirable effects. Intakes of 1,000 mg or more of vitamin C can be consumed daily without any known adverse effects.

Ascorbic acid is the preferred source of vitamin C in a composition of the present invention, although other sources such as for example sodium ascorbate can alternatively be used.

Alpha-Tocopherol

Alpha-tocopherol or vitamin E is also a well-known antioxidant. Vitamin E can work synergistically with vitamin C in protecting vital cell function from normal oxidants. Vitamin E is a relatively non-toxic fat-soluble vitamin. Vitamin E is readily oxidized thereby significantly reducing its activity during periods of storage prior to ingestion, and this degradation should be taken into account in the formulation of a nutritional or dietary composition of the present invention. Once ingested, vitamin E is stored within the body and can contribute to the total body pool of vitamin E for up to one year.

Vitamin E is the major chain breaking lipid-soluble antioxidant in membranes. It scavenges free radicals, particularly hydroxyl radical and singlet oxygen. B. S. Winkler et al., Molecular Vision, Vol. 5, 32 (1999). It acts by at least two different mechanisms: by directly scavenging reactive oxygen species (“ROS”) and by up-regulating antioxidant enzymes such are glutathione peroxidase (“GPX”), superoxide dysmutase (“SOD”), catalase (“CAT”), and glutathione reductase (“GR”). S. Vertuani et al., Current Pharm. Des., Vol. 10, 1677 (2004). The increased oxidative stress and impaired antioxidant defense associated with hyperglycemia are also related to the oxidative damage of LDL and to the elevated lipid peroxide levels in blood. Lipid peroxidation is an initial event within LDL oxidative modification induced by the ROS and nitrogen species. The oxidative modification of LDL seems to be a key event in atherosclerosis induction and/or progression. According to the oxidation theory, the formation of oxidized LDL (oxLDL) in the subendothelial space thought to play a causative role, and antioxidants are therefore potential anti-atherogenic compounds. The oxLDL has many pro-atherogenic activities. They can stimulate the expression of endothelial adhesion molecules, have chemotactic effect and inhibit the migration of macrophages outside the subendothelial space, thus increasing the number of leukocytes and proinflammatory factors involved in atherogenesis. R. Stocker, Trends Biochem., Vol. 24, 219 (1999). α-Tocopherol, among the eight different forms of vitamin E, is selectively retained in the body and secreted by the liver as an integral component of VLDL, the precursor of LDL. It is the most abundant and active scavenger of peroxyl radicals present within LDL. α-Tocopherol acts by inhibiting the radical chain propagation within lipid domains thus leading to stable lipid species. The ability of α-tocopherol to inhibit the in vitro LDL oxidation is unequivocally proven. This observation has suggested the possibility that vitamin E may reduce the occurrence of atheosclerotic lesions through the prevention of the initial oxidative event. A. Munteanu et al., J. Cell Mol. Med., Vol. 8, 59 (2004).

The RDA of vitamin E in the form of DL-alpha tocopheryl acetate is 30 IU. No adverse effects of DL-α-tocopheryl acetate have been observed at levels as high as 800 mg, with 1 mg of dl-α-tocopheryl acetate being equal to 1 IU of DL-α-tocopheryl acetate. See U.S. Pat. No. 6,660,297. DL-α-Tocopheryl acetate is one suitable source of vitamin E in a composition of the present invention although other sources of vitamin E, such as for example trimethyl tocopheryl acetate and/or vitamin E succinate, may be used in the alternative.

Alpha-tocopherol included in a composition of the present invention can be in the form of α-tocopheryl nicotinate, α-tocopheryl phosphate, α-tocopheryl succinate, α-tocopheryl acetate, including an isomer thereof, or a racemic mixture thereof. In one embodiment, a composition of the present invention includes D-α-tocopheryl succinate. In another embodiment, a composition of the present invention includes DL-α-tocopheryl acetate.

### Vitamin B₁

Evidence is now emerging that vitamin B₁ (thiamine and benfotiamine, a lipophilic derivative of thiamine-monophosphate) is able to prevent the development of microvascular complications of diabetes. By investigating the effect of these agents in diabetic rats it has been found that these animals have abnormally low plasma vitamin B₁ concentration, probably associated with its increased urinary excretion and decreased thiamine reabsorption in renal tubules. Clinical diabetes is also associated with a mild vitamin B₁ deficiency. Two studies have found 18 and 76%, respectively of diabetic subjects studied to have plasma thiamine concentration lower than the normal range minimum. N. Saito et al., J. Nutr. Sci. Vitaminol., Vol. 33, 421 (1987); E. Havivi et al., Int. J. Nutr. Res., Vol. 61, 328 (1991). In addition, recent studies show that thiamine and benfotiamine are effective in reducing AGE generation in human endothelial cell cultures (F. Pomero et al., Acta Diabetol., Vol. 38, 135 (2001)) and in preventing AGE accumulation in the retina of diabetic rats (N. Karachalias et al., Ann. NY Acad. Sci., Vol. 1043, 777 (2005)).

Thiamine has an important role in the metabolism of glucose and the daily requirement for this vitamin is related to energy need, particularly that which is derived from carbohydrate. It has been found that 0.33 mg of thiamine is required for each 4400 kJ of energy requirement. The Food and Nutrition Board of the National Research Council therefore recommends a thiamine intake of 0.5 mg/4400 kJ and considers this will maintain a satisfactory vitamin/carbohydrate balance. It has also been recommended that because elderly people may use thiamine less efficiently, their supplementary intake should be 1 mg/day regardless of their dietary intake. R. E. Davis and G. C. Icke, Adv. Clin. Chem., Vol 23, 93 (1983).

No adverse side effects are known with thiamine intakes at RDA levels or even at levels several times the RDA. The RDA of vitamin B₁ in most countries is 1.4 mg.

Vitamin B₁ included in a composition of the present invention can be in the form of thiamine hydrochloride, thiamine monophosphate hydrochloride dehydrate, thiamine nitrate, or benfotiamine monophosphate.

### Vitamin B₃

Vitamin B₃ or niacin (or nicotinic acid) is a water-soluble vitamin. The designation “vitamin B₃” also includes niacinamide (or also known as nicotinamide), which has identical vitamin activities, but very different pharmacological activities. Niacinamide, via its major metabolites NAD⁺ and NADP⁺, is involved in a wide range of biological processes, including the production of energy, the synthesis of fatty acids, cholesterol, and steroids, signal transduction, and the maintenance of the integrity of the genome. Nicotinic acid, in pharmacological doses, is used as an antihyperlipidemic agent. C. Bourgeois, Modern Nutrition in Health and Disease, 10^{th} Ed, Lippincott Williams & Wilkins (2005).

The mechanism of action of niacinamide is dual: it is an essential part of NAD, thus it may prevent NAD depletion, typically occurring in diabetes, by increasing the NAD pool, and it acts as poly(ADP-ribose) polymerase-1 (“PARP-1”) inhibitor. In diabetes, oxidative stress plays a key role in the pathogenesis of vascular complications, and an early step of such damage is considered to be the development of an endothelial dysfunction. Hyperglycemia directly promotes an endothelial dysfunction inducing process of overproduction of superoxide and consequently peroxynitrite that damages DNA and activates PARP-1. PARP-1 is a nuclear enzyme that is activated by oxidant-induced DNA single-strand breakage and transfers ADP-ribose residues from NAD⁺ to nuclear proteins. PARP-1 activation is clearly manifest in diabetes and contributes to diabetic endothelial dysfunction. F. Garcia-Soriano et al., Nat. Med., Vol. 7, 108 (2001), P. Pacher et al., Diabetes, Vol. 51, 514 (2002). PARP-1 has been implicated in DNA repair, control of genome integrity, apoptosis, and NF-κB regulation. S. Shall et al., Mutat. Res., Vol. 460, 1 (2000); M. Kameoka et al., J. Biochem., Vol. 346, 641 (2000); Z. Herceg et al., Mutat. Res., Vol. 477, 97 (2001). Furthermore, antioxidant effect of niacinamide has been shown in animal models in which levels of oxidative biomarkers were reduced following niacinamide administration. M. J. Stevens et al., J. Pharmacol Exp. Ther. Vol. 320, 458 (2006); A. Kretowski et al., Horm. Metab. Res. Vol. 28, 35 (1996).

The risk assessment for high dose niacinamide has been carefully reviewed. In short- and long-term studies niacinamide has been found to have low toxicity. No carcinogenic effects have been demonstrated by long-term feeding of niacinamide to rats (1% in drinking water). There is no evidence of teratogenic effects. There have been some clinical reports of adverse effects in humans taking large doses of niacinamide for a long time, in particular of readily-reversed increment in serum levels of liver enzymes. It is suggested that niacinamide at doses up to a maximum of 3 g/day could achieve partial PARP inhibition in humans and its safety profile seemed good. F. Pociot, IDIG Workshop, Copenhagen, Denmark, 4-5 Dec. 1992, Diabetologia 1993.

The RDA of niacin is 2-12 mg for children and 14-16 mg for adults.

### Vitamin B₆

The vitamin B₆ family consists of 3 members: pyridoxamine (“PM”), pyridoxine (“PN”), pyridoxal (“PL”). It is known, both from in vitro and in vivo studies, that PM is a potent inhibitor of the formation of AGE from Amadori adducts (R. G. Khalifah et al., Biochem. Biophys. Res. Commun., Vol. 257, 251 (1999)) and is able to limit the formation of proteins modified chemically by carbonyls and by lipids. T. O. Metz et al., Arch. Biochem. Biophys., Vol. 419, 41 (2003). For these reasons, new interest has arisen regarding the possibility to use this B₆ vitamer as a prospective pharmacological agent for treatment of complications of diabetes. Actually, PM has a unique mechanism of action different than that of PN, and PN cannot be transformed into PM. However, PN has been included in the dietary supplement because a deficiency of PN in diabetic animals as well as subjects has been reported. M. Okada et al., Diabetes Obes. Metab. Vol. 1, 221 (1999); J. M. Ellis et al., Biochem. Biophys. Res. Comm. Vol. 179, 615 (1991).

The U.S. RDA varies between 1.3 and 2 mg, depending upon age and gender.

Other ingredients believed to be of benefit in maintaining, maintaining, strengthening, improving, or promoting eye health; especially, that of diabetic patients, may likewise be added to a nutritional or dietary composition of the present invention, if desired. Such ingredients include for example but are not limited to β-carotene, lutein, zeaxanthine, lycopene, astaxanthin, flavonoids, resveratrol, phenolic compounds (such as, for example, oligomeric proanthocyanidins), anthocyanosides, essential fatty acids (such as omega-3 or omega-6), nutritional minerals and/or metals, and combinations thereof as is discussed in more detail below.

### Beta-Carotene

Beta-carotene, a proform of vitamin A, is a lipid-soluble orange pigment found in many vegetables. Beta-carotene is converted to vitamin A in the body with an efficiency of approximately 50 percent. The RDA of vitamin A is 5,000 IU. Beta-carotene has one of the highest antioxidant potentials of the antioxidants. No observed adverse effects are observed for beta-carotene at dosage levels as high as 25 mg per day for healthy, non-smokers. However, an increased risk of fatal coronary heart attacks in men with previous myocardial infarction and an increased risk of lung cancer among male smokers have been observed in individuals who receive 20 mg/day of β-carotene. See U.S. Pat. No. 6,660,297. Preferably, each tablet of a four tablet per day dosage regime of the subject composition provides not less than approximately 4.3 mg, but more preferably approximately 6 mg, of β-carotene. Such a formulation provides a total daily dosage of preferably not less than approximately 17.2 mg, but more preferably approximately 24 mg, of β-carotene, and preferably, not more than approximately 28 mg β-carotene. At a potency of 1,667 IU vitamin A per mg β-carotene, this daily dosage of β-carotene is equivalent to approximately 6 to 10 times the RDA of vitamin A.

Beta-carotene is preferred in a composition of the present invention due to its ready commercial availability although alternative carotenoid proforms of vitamin A could likewise be used. A product comprising β-carotene would not be recommended for smokers because of its high potency.

### Lutein

Lutein, like β-carotene, is a carotenoid. Lutein is one of the most abundant carotenoids found in fruits and vegetables. Lutein is also an antioxidant found in the retina of healthy eyes. Preferably, each tablet of a four tablet per day dosage regime could provide approximately 0.25 to 10 mg of lutein for a total daily dosage of approximately 1 to 40 mg depending upon whether lutein is used to supplement or substitute β-carotene and/or zeaxanthin.

### Zeaxanthin

Zeaxanthin, like lutein and β-carotene, is a carotenoid. Zeaxanthin is found naturally in fruits and vegetables. Zeaxanthin is also an antioxidant found in the retina of healthy eyes. Preferably, each tablet of a four tablet per day dosage regime could provide approximately 0.01 to 10 mg of zeaxanthin for a total daily dosage of approximately 0.04 to 40 mg depending upon whether zeaxanthin is used to supplement or substitute beta-carotene and/or lutein. As with beta-carotene, zeaxanthin is subject to degradation during periods of storage prior to ingestion. Accordingly, larger quantities of zeaxanthin are necessary in a tablet than the desired daily dosage quantity of zeaxanthin to be provided upon ingestion.

### Lutein-Zeaxanthin

Lutein-zeaxanthin raw material combinations achieved deliberately, because of normal composition, or through raw material contamination may likewise be added to the subject composition as desired. Preferred ratios of lutein-zeaxanthin for example include 90 to 99 percent lutein and 1 to 10 percent zeaxanthin or 90 to 99 percent zeaxanthin and 1 to 10 percent lutein. Preferably, each tablet of a four tablet per day dosage regime could provide approximately 0.01 to 10 mg of lutein-zeaxanthin for a total daily dosage of approximately 0.04 to 40 mg depending upon whether lutein-zeaxanthin is used to supplement or substitute β-carotene.

### Phenolic Compounds

Phenolic compounds such as for example but not limited to oligomeric proanthocyanidins are additional useful antioxidants. Oligomeric proanthocyanidins are found naturally in grape seeds. Phenolic compounds may be added to the nutritional or dietary supplement composition of the present invention if desired. If so desired, preferably each tablet of a four tablet per day dosage regime would provide approximately 0.25 to 5 mg of phenolic compounds for a total daily dosage of approximately 1 to 20 mg.

### Anthocyanosides

Anthocyanosides are useful antioxidants found naturally in bilberry fruit. Anthocyanosides may be added to the nutritional or dietary supplement composition of the present invention if desired. If so desired, preferably each tablet of a four tablet per day dosage regime would provide approximately 0.25 to 5 mg of anthocyanosides for a total daily dosage of approximately 1 to 20 mg.

### Lycopene

Lycopene is an oil-soluble member of the class of carotenes of the carotenoid pigments found in tomatoes and other red fruits. It is one of the most potent carotenoid antioxidants and the most powerful carotenoid quencher of singlet oxygen. Singlet oxygen from ultraviolet light is a primary cause of skin aging due to its powerful oxidation potential. There is evidence that frequent intake of lycopene is associated with reduced risk of diabetes as measured by impaired glucose tolerance. Ford et al., Am. J. Epidemiol., Vol. 149, 168 (1999). Lycopene has also been found to possess antiproliferative properties in animal and in vitro studies. D. Heber et al., Exp. Biol. Med., Vol. 227, 920 (2002); E. Kotake-Nara et al., J. Nutr., Vol. 131, 3303 (2001). The average daily intake of lycopene is 25 mg. A. V. Rao et al., J. Am. Coll. Nutr., Vol. 19, NO. 5, 563 (2000). Lycopene has a low order of toxicity with a large margin of safety. M. McClain et al., Regul. Toxicol. Pharmacol., Vol. 37, 274 (2003). No tolerable upper limit for lycopene intake has been established.

### Astaxanthin

Astaxanthin is a member of the xanthophylls of the carotenoid pigments. It is a powerful antioxidant, having 100-500 times the antioxidant capacity of vitamin E and 10 times the antioxidant capacity of β-carotene. Several studies have indicated that astaxanthin is a stronger antioxidant than lutein, lycopene, or tocotrienols (see http://www.beta-glucan-info.com/astaxanthin-questions-answers.htm, visited Jan. 25, 2007). A recent study showed that a daily dose of 19.25 mg administered over a period of 29 days to healthy humans had no ill effects (see http://www.astaxanthin.org/humansafety.htm, visited Jan. 25, 2007).

### Flavonoid

Flavonoids are a class of plant secondary metabolites and have the common 2-phenyl-1,4-benzopyrone structure. They are commonly known for their antioxidant activity. There has been strong evidence of their inherent ability to modify the body's reaction to allergens, viruses, and carcinogens. They also show anti-allergic, anti-inflammatory, anti-microbial, and anti-cancer activity. Good sources of flavonoids include all citrus fruits, berries, parsley, legumes, onions, green tea, and red wine. Average daily intake is in the range 50-800 mg (see http://lpi.oregonstate.edu/f-wO0/flavonoid.html, visited Jan. 12, 2007). No tolerable upper limit has been established.

### Resveratrol

Resveratrol is a phytoalexin produced by several plant species, apparently for its antifungal properties. It is found in grapes (primarily the skins), raspberries, mulberries, blueberries, bilberries, cranberries, and some pines. See; e.g., http://lpi.oregonstate.edu/infocenter/phytochemicals/resveratrol/ (visited Jan. 25, 2005). No ill side effects were found for a daily dose of equal to or less than 5 g in a human clinical trial. D. J. Boocook et al., Proc. Am. Assoc. Cancer Res., Vol. 47, Abstract #5741 (2006).

### Chromium(III)

Chromium is a mineral that humans require in trace amount, although its mechanism of action in the body and the amount need for optimal health are not well defined. Chromium(III) is biologically active and found in food. Some studies have shown that chromium enhances the action of insulin, a hormone critical to the metabolism and storage of carbohydrate, fat, and protein in the body. W. Mertz, Physiol. Rev., Vol. 49, 163 (1969); W. Mertz, J. Nutr., Vol. 123, 626 (1993); W. Mertz, Nutr. Rev., Vol. 56, 174 (1998); D. Porter, Jr. et al., Ellengerg & Rifkin's Diabetes Mellitus, A. Baron (Editor), 6th Ed., McGraw-Hill (2003). Chromium also appears to be directly involved in carbohydrate, fat, and protein metabolism. However, more research is needed to determine the full range of its role in the body. Thus, it can be gleaned from published research studies that chromium deficiency can impair the body's ability to use glucose to meet its energy needs and raise insulin requirements. Chromium supplements, therefore, can be useful to help to control type-2 diabetes or the glucose and insulin responses in persons at risk of developing this disease and complications, including ocular neovascularization, resulting therefrom.

The Food and Nutrition Board (“FNB”) of the Institute of Medicine has not set a tolerable upper level of intake for chromium. The RDA for chromium is 11 μg/day for children of age 1-3 years, 15 μg/day for children of age 4-8 years, and 25-30 μg/day for adults. A study of 10 women taking 400 μg/day of chromium as chromium(III) picolinate found no evidence of increased oxidative damage to DNA as measured by antibodies to an oxidized DNA base. I. Kato et al., Eur. J. Epidemiol., Vol. 14, No. 6, 621 (1998).

In one aspect, a daily dose of a composition of the present invention can comprise from about 50 to about 500 μg chromium(III). Alternatively, a daily dose of a composition of the present invention can comprise from about 100 to about 300 μg, or from about 150 to about 250 μg, or from about 50 to about 100 μg, or from about 50 to about 200 μg. Chromium(III) included in a composition of the present invention can be in the form of, for example, chromium(III) acetate, chromium(III) chloride, chromium(III) chloride hexahydrate, chromium(III) chloride hydrate, chromium(III) nitrate monohydrate, chromium(III) phosphate hydrate, chromium(III) sulfate, chromium(III) picolinate, chromium(III) nicotinate, or chromium(III) histidinate.

The ingredients of a nutritional or dietary supplement composition of the present invention, considered individually, have been known to provide certain physiological effects. However, the unique formulations and the effects thereof on eye health have not previously been known or could not have been predicted.

In one aspect, pharmaceutically acceptable inactive ingredients well known in the art can be included in a composition of the present invention to aid in manufacturing said composition in various forms. For example, inactive ingredients may include but are not limited to excipients, carriers, diluetnts, binders, lubricants, disintigrants, and mixtures thereof, such as for example, cellulose, gelatin, magnesium stearate, water, vegetable oil, glycerin, beeswax and silica. The particular carrier, diluent or excipient used will depend upon the means and purpose for which the active ingredient is being applied.

In one embodiment, a tablet formulation includes materials such as diluents, binders, lubricants, disintegrants, and mixtures thereof. Suitable diluents include various types of starch, lactose, mannitol, kaolin, calcium phosphate or sulfate, inorganic salts (e.g., sodium chloride), powdered sugar, and powdered cellulose derivatives. Non-limiting examples of diluents are microcrystalline cellulose (e.g., Avicelt PH102 or PH101 available from FMC Pharmaceutical, Philadelphia, Pa.) and lactose. The mean particle size for the microcrystalline cellulose generally ranges from about 90 μm to about 200 μm. Suitable grades of lactose include anhydrous lactose (about 152 μm mean particle size), lactose monohydrate and spray dried lactose (e.g., Fast Flo® lactose, about 87 μm mean particle size, available from Foremost Corp., Baraboo, Wis.).

If desired, a binder may be added. Suitable binders include substances such as celluloses (e.g., cellulose, methylcellulose, ethylcellulose, and hydroxymethylcellulose), polypropylpyrrolidone, polyvinylpyrrolidone, gelatin, gum arabic, polyethylene glycol, starch, sugars (e.g., lactose, sucrose, fructose, and glucose), natural and synthetic gums (e.g., acacia, alginates, and gum arabic) and waxes.

A lubricant is typically used in a tablet formulation to prevent the tablet and punches from sticking in the die. Suitable lubricants include slippery solids such as talc, magnesium and calcium stearate, stearic acid, light anhydrous silicic acid, and hydrogenated vegetable oils. A preferred lubricant is magnesium stearate.

Disintegrants may also be added to the composition to break up the dosage form and release the compound. Suitable disintegrants include starches (e.g., corn or potato starches and hydroxypropylstarch), clays, celluloses (e.g., cellulose, wood cellulose, methyl- or ethyl-cellulose, substituted hydroxypropylcellulose, and carboxymethylcellulose), agar, algins (e.g., alginic acid), powdered natural sponge, cation-exchange resins, citrus pulp, bentonite, sodium bicarbonate, calcium phosphate, calcium citrate, sodium lauryl sulfate, and gums (e.g., guar gum).

Other useful additives include materials such as agents for retarding dissolution (e.g., paraffin), resorption accelerators (e.g., quaternary ammonium compounds), surface active agents (e.g., cetyl alcohol, glycerol monostearate, and sodium lauryl sulfate), adsorptive carriers (e.g., kaolin and bentonite), preservatives, sweeteners, coloring agents, flavoring agents (e.g., citric acid, menthol, glycine or orange powder), stabilizers (e.g., citric acid or sodium citrate), binders (e.g., hydroxypropylmethylcellulose), and mixtures thereof.

A safe and effective method of preventing, stabilizing, reversing and/or treating complications of diabetic retinopathy, diabetic macular edema, cataract, glaucoma, or ocular inflammation (such as uveitis) comprises providing a human or other animal a daily dosage that comprises 50-1000 mg α-lipoic acid, 10-200 mg genistein, 50-1000 mg ascorbic acid, and 10-1000 mg α-tocopherol.

In one aspect, one or more ingredients of said daily dosage are substituted with one or more of its pharmaceutically acceptable derivatives to provide a total therapeutically equivalent amount of each ingredient.

In another aspect, a formulation that is administered to said human or animal at a frequency of one, two, four, or more times per day such that the daily dosage is achieved.

A method of manufacturing a nutritional or dietary supplement composition of the present invention, which is safe and effective in the prevention, stabilization, reversal and/or treatment of complications of diabetic retinopathy, diabetic macular edema, cataract, glaucoma, or ocular inflammation (such as uveitis), includes providing at least α-lipoic acid, genistein, ascorbic acid, and α-tocopherol. These ingredients, as well as any desired inactive ingredients (such as pharmaceutically acceptable carriers) and/or additional ingredients are combined in quantities as described above and mechanically combined, such as for example, through the use of a blender to form a blend. If necessary, the blend is then further mixed until substantial uniformity is achieved. The blend is then formed into articles, such as tablets, caplets, or capsules. Alternatively, the blend can be in the form of syrup, solution, dispersion, emulsion, or gel.

In one embodiment, the blend is compressed using a tablet press to form tablets. Optionally, a coating may be sprayed on the tablets and the tablets tumbled until dry. Alternatively, the blend may be placed in mineral oil to form a slurry for containment in a soft gel capsule, the blend may be placed in a gelatin capsule or the blend may be placed in other dosage forms known to those skilled in the art.

### EXAMPLE 1

A composition in the form of a tablet to be taken orally by a person once per day comprises the ingredients in amounts shown in Table I.

TABLE I

| Ingredient | Amount | | Preferred Amount | |
|---|---|---|---|---|
| α-lipoic acid | 300-450 | mg | 300-450 | mg |
| genistein | 50-70 | mg | 50-70 | mg |
| ascorbic acid | 200-300 | mg | 200-290 | mg |
| DL-α-tocopheryl acetate | 200-350 | mg | 200-330 | mg |
| thiamine mononitrate | 1.5-3.5 | mg | 1.8-3.2 | mg |
| niacinamide | 15-30 | mg | 20-30 | mg |
| pyridoxine hydrochloride | 2-4 | mg | 2.5-3.7 | mg |

### EXAMPLE 2

A composition in the form of a tablet to be taken orally by a person twice per day comprises the ingredients in amounts shown in Table II, for a total of two tablets per day.

TABLE II

| Ingredient | Amount | | Preferred Amount | |
|---|---|---|---|---|
| α-lipoic acid | 150-225 | mg | 150-225 | mg |
| genistein | 25-35 | mg | 25-35 | mg |
| ascorbic acid | 100-150 | mg | 100-145 | mg |
| DL-α-tocopheryl acetate | 100-175 | mg | 100-165 | mg |
| thiamine mononitrate | 0.75-1.75 | mg | 0.75-1.6 | mg |
| niacinamide | 7.5-15 | mg | 10-15 | mg |
| pyridoxine hydrochloride | 1-2 | mg | 1.25-1.85 | mg |

### EXAMPLE 3

A composition in the form of a caplet to be taken orally by a person once per day comprises the ingredients in amounts shown in Table III.

TABLE III

| Ingredient | Amount | | Preferred Amount | |
|---|---|---|---|---|
| α-lipoic acid | 300-450 | mg | 300-450 | mg |
| genistein | 50-70 | mg | 50-70 | mg |
| ascorbic acid | 200-300 | mg | 200-290 | mg |
| DL-α-tocopheryl acetate | 200-350 | mg | 200-330 | mg |
| thiamine mononitrate | 1.5-3.5 | mg | 1.8-3.2 | mg |
| niacinamide | 15-30 | mg | 20-30 | mg |
| pyridoxine hydrochloride | 2-4 | mg | 2.5-3.7 | mg |
| chromium(III) chloride | 100-250 | μg | 120-240 | μg |
| hexahydrate | | | | |

### EXAMPLE 4

A composition in the form of a caplet to be taken orally by a person twice per day comprises the ingredients in amounts shown in Table IV, for a total of two caplets per day.

TABLE IV

| Ingredient | Amount | | Preferred Amount | |
|---|---|---|---|---|
| α-lipoic acid | 150-225 | mg | 150-225 | mg |
| genistein | 25-35 | mg | 25-35 | mg |
| ascorbic acid | 100-150 | mg | 100-145 | mg |
| DL-α-tocopheryl acetate | 100-175 | mg | 100-165 | mg |
| thiamine mononitrate | 0.75-1.75 | mg | 0.9-1.6 | mg |
| niacinamide | 7.5-15 | mg | 10-15 | mg |
| pyridoxine hydrochloride | 1-2 | mg | 1.5-1.85 | mg |
| chromium(III) chloride | 50-125 | μg | 60-120 | μg |
| hexahydrate | | | | |

### EXAMPLE 5

A composition in the form of a capsule to be taken orally by a person once per day comprises the ingredients in amounts shown in Table V.

TABLE V

| Ingredient | Amount | | Preferred Amount | |
|---|---|---|---|---|
| α-lipoic acid | 300-450 | mg | 300-450 | mg |
| genistein | 50-70 | mg | 50-70 | mg |
| ascorbic acid | 200-300 | mg | 200-290 | mg |
| DL-α-tocopheryl acetate | 200-350 | mg | 200-330 | mg |
| thiamine mononitrate | 1.5-3.5 | mg | 1.8-3.2 | mg |
| niacinamide | 15-30 | mg | 20-30 | mg |
| pyridoxine hydrochloride | 2-4 | μg | 2.5-3.7 | mg |
| chromium(III) picolinate | 120-250 | μg | 120-240 | μg |

### EXAMPLE 6

A composition in the form of a tablet to be taken orally by a person twice per day comprises the ingredients in amounts shown in Table VI, for a total of two tablets per day.

TABLE VI

| Ingredient | Amount | | Preferred Amount | |
|---|---|---|---|---|
| α-lipoic acid | 150-225 | mg | 150-225 | mg |
| genistein | 25-35 | mg | 25-35 | mg |
| ascorbic acid | 100-150 | mg | 100-145 | mg |
| DL-α-tocopheryl acetate | 100-175 | mg | 100-165 | mg |
| thiamine mononitrate | 0.75-1.75 | mg | 0.9-1.6 | mg |
| niacinamide | 7.5-15 | mg | 10-15 | mg |
| pyridoxine hydrochloride | 1-2 | mg | 1.25-1.85 | mg |
| chromium(III) picolinate | 60-125 | μg | 60-120 | μg |

### EXAMPLE 7

A composition in the form of a tablet to be taken orally by a person twice per day comprises the ingredients in amounts shown in Table VII, for a total of two tablets per day.

TABLE VII

| Ingredient | Amount | |
|---|---|---|
| α-lipoic acid | 150-225 | mg |
| genistein | 25-35 | mg |
| ascorbic acid | 100-145 | mg |
| DL-α-tocopheryl acetate | 100-165 | mg |
| thiamine mononitrate | 0.7-2 | mg |
| niacinamide | 10-15 | mg |
| pyridoxine hydrochloride | 1-2 | mg |
| chromium(III) picolinate | 60-120 | μg |
| lutein | 0.5-20 | mg |

### EXAMPLE 8

A composition in the form of a caplet to be taken orally by a person twice per day comprises the ingredients in amounts shown in Table VIII, for a total of two caplets per day.

TABLE VIII

| Ingredient | Amount | |
|---|---|---|
| α-lipoic acid | 150-225 | mg |
| genistein | 25-35 | mg |
| ascorbic acid | 100-145 | mg |
| α-tocopheryl acetate | 100-165 | mg |
| thiamine mononitrate | 0.7-2 | mg |
| niacinamide | 10-15 | mg |
| pyridoxine hydrochloride | 1-2 | mg |
| chromium(III) picolinate | 60-120 | μg |
| zeaxanthine | 0.02-20 | mg |

### EXAMPLE 9

A composition in the form of a capsule to be taken orally by a person four times per day comprises the ingredients in amounts shown in Table IX, for a total of four capsules per day.

TABLE IX

| Ingredient | Amount | |
|---|---|---|
| α-lipoic acid | 35-60 | mg |
| genistein | 6-9 | mg |
| ascorbic acid | 50-75 | mg |
| α-tocopheryl acetate | 50-85 | mg |
| thiamine mononitrate | 0.4-1 | mg |
| niacinamide | 5-7 | mg |
| pyridoxine hydrochloride | 0.5-1 | mg |
| chromium(III) picolinate | 30-60 | μg |
| lutein | 0.3-10 | mg |
| grape seed oil (containing | 0.5-10 | mg |
| proanthocyanidin) | | |

### EXAMPLE 10

A composition in the form of a capsule to be taken orally by a person four times per day comprises the ingredients in amounts shown in Table X, for a total of four capsules per day.

TABLE X

| Ingredient | Amount | |
|---|---|---|
| α-lipoic acid | 75-100 | mg |
| genistein | 12-20 | mg |
| ascorbic acid | 50-70 | mg |
| α-tocopheryl acetate | 50-70 | mg |
| thiamine mononitrate | 0.4-0.7 | mg |
| niacinamide | 5-7 | mg |
| pyridoxine hydrochloride | 0.5-1 | mg |
| chromium(III) picolinate | 30-60 | μg |
| lutein | 0.3-10 | mg |
| bilberry extract (containing | 0.5-15 | mg |
| anthocyanosides) | | |

### EXAMPLE 11

A composition in the form of a tablet to be taken orally by a person twice per day comprises the ingredients in amounts shown in Table XI, for a total of two tablets per day.

TABLE XI

| Ingredient | Amount | | Preferred Amount | |
|---|---|---|---|---|
| α-lipoic acid | 50-120 | mg | 65-155 | mg |
| genistein | 5-30 | mg | 5-25 | mg |
| ascorbic acid | 35-75 | mg | 45-70 | mg |
| α-tocopheryl acetate | 5-13 | mg | 6-12 | mg |
| thiamine mononitrate | 0.5-1.5 | mg | 0.75-1.35 | mg |
| niacinamide | 7-15 | mg | 9-14 | mg |
| pyridoxine hydrochloride | 0.7-2 | mg | 1.1-1.75 | mg |
| chromium(III) chloride | 0.1-0.4 | mg | 0.15-0.3 | mg |

### EXAMPLE 12

A composition in the form of a tablet to be taken orally by a person once per day comprises the ingredients in amounts shown in Table XII.

TABLE XII

| Ingredient | Amount | | Preferred Amount | |
|---|---|---|---|---|
| α-lipoic acid | 100-240 | mg | 135-210 | mg |
| genistein | 10-60 | mg | 10-50 | mg |
| ascorbic acid | 70-150 | mg | 90-140 | mg |
| DL-α-tocopheryl acetate | 10-25 | mg | 12-24 | mg |
| thiamine mononitrate | 1-3 | mg | 1.5-2.7 | mg |
| niacinamide | 15-30 | mg | 18-28 | mg |
| pyridoxine hydrochloride | 1.5-4 | mg | 2.2-3.5 | mg |
| chromium(III) chloride | 0.2-0.7 | mg | 0.3-0.6 | mg |

### EXAMPLE 13

A composition in the form of a tablet to be taken orally by a person once per day comprises the ingredients in amounts shown in Table XIII.

TABLE XIII

| Ingredient | Amount | | Preferred Amount | |
|---|---|---|---|---|
| α-lipoic acid | 300-450 | mg | 300-450 | mg |
| ascorbic acid | 200-300 | mg | 200-290 | mg |
| DL-α-tocopheryl acetate | 200-350 | mg | 200-330 | mg |
| thiamine mononitrate | 1.5-3.5 | mg | 1.8-3.2 | mg |
| niacinamide | 15-30 | mg | 20-30 | mg |
| pyridoxine hydrochloride | 2-4 | mg | 2.5-3.7 | mg |

### EXAMPLE 14

A composition in the form of a tablet to be taken orally by a person once per day comprises the ingredients in amounts shown in Table XIV.

TABLE XIV

| Ingredient | Amount | | Preferred Amount | |
|---|---|---|---|---|
| α-lipoic acid | 100-240 | mg | 135-210 | mg |
| ascorbic acid | 70-150 | mg | 90-140 | mg |
| DL-α-tocopheryl acetate | 10-25 | mg | 12-24 | mg |
| thiamine mononitrate | 1-3 | mg | 1.5-2.7 | mg |
| niacinamide | 15-30 | mg | 18-28 | mg |
| pyridoxine hydrochloride | 1.5-4 | mg | 2.2-3.5 | mg |
| chromium(III) chloride | 0.2-0.7 | mg | 0.3-0.6 | mg |

In another aspect, each of the compositions of Examples 1-14 further comprises a pharmaceutically acceptable carrier.

In still another aspect, a composition of the present invention consists essentially of the ingredients shown in any of Tables I-XIV, a pharmaceutically acceptable carrier, and a pharmaceutically acceptable diluent.

In still another aspect, a composition of the present invention consists essentially of the ingredients in the amounts shown in Table 1 or 2 and a pharmaceutically acceptable carrier.

In still another aspect, a composition of the present invention consists essentially of the ingredients in the amounts shown in Table 1 or 2, a pharmaceutically acceptable carrier, and a pharmaceutically acceptable diluent.

In yet another aspect, a composition of the present invention consists essentially of the ingredients in the amounts shown in Table 1 or 2; at least a material selected from the group consisting of β-carotene, lutein, zeaxanthine, lycopene, astaxanthin, flavonoids, resveratrol, phenolic compounds (such as, for example, oligomeric proanthocyanidins), anthocyanosides, and essential fatty acids (such as omega-3 or omega-6); and a pharmaceutically acceptable carrier.

In a further aspect, a composition of the present invention consists essentially of the ingredients in the amounts shown in Table 1 or 2; at least a material selected from the group consisting of β-carotene, lutein, zeaxanthine, lycopene, astaxanthin, flavonoids, resveratrol, phenolic compounds (such as, for example, oligomeric proanthocyanidins), anthocyanosides, and essential fatty acids (such as omega-3 or omega-6); an essential mineral or metal; and a pharmaceutically acceptable carrier.

In still a further aspect, said essential mineral or metal is selected from the group consisting of chromium(III) and pharmaceutically acceptable salts thereof.

In still another aspect, In a further aspect, a composition of the present invention consists essentially of the ingredients in the amounts shown in Table 1 or 2; at least a material selected from the group consisting of β-carotene, lutein, zeaxanthine, lycopene, astaxanthin, flavonoids, resveratrol, phenolic compounds (such as, for example, oligomeric proanthocyanidins), anthocyanosides, and essential fatty acids (such as omega-3 or omega-6); an essential mineral or metal; a pharmaceutically acceptable carrier; and a pharmaceutically diluent.

While specific embodiments of the present invention have been described in the foregoing, it will be appreciated by those skilled in the art that many equivalents, modifications, substitutions, and variations may be made thereto without departing from the spirit and scope of the invention as defined in the appended claims.

What is claimed is:

1. A nutritional or dietary composition comprising, on a daily dosing basis: (a) α-lipoic acid or a pharmaceutical equivalent thereof; (b) ascorbic acid or a pharmaceutical equivalent thereof; and (c) α-tocopherol or a pharmaceutical equivalent thereof.

2. The composition of claim 1, wherein α-lipoic acid or a pharmaceutical equivalent thereof is present in an amount of about 50-1000 mg, ascorbic acid or a pharmaceutical equivalent thereof is present in an amount of about 50-1000 mg, and α-tocopherol or a pharmaceutical equivalent thereof is present in an amount of about 10-1000 mg.

3. The composition of claim 2, further comprising genistein or a pharmaceutical equivalent thereof in an amount of about 20-200 mg.

4. The composition of claim 1, wherein α-lipoic acid or a pharmaceutical equivalent thereof is present in an amount of about 100-600 mg, ascorbic acid or a pharmaceutical equivalent thereof is present in an amount of about 100-600 mg, and α-tocopherol or a pharmaceutical equivalent thereof is present in an amount of about 10-600 mg.

5. The composition of claim 2, further comprising genistein or a pharmaceutical equivalent thereof in an amount of about 20-100 mg,

6. The composition of claim 3, further comprising a material selected from the group consisting of vitamin B₁, vitamin B₃, vitamin B₆, pharmaceutical equivalents thereof, and combinations thereof.

7. The composition of claim 6, wherein said material is present in an amount from about 100% to about 300% of an RDA thereof.

8. The composition of claim 6, wherein said material is present in an amount from about 100% to about 200% of an RDA thereof.

9. The composition of claim 7, further comprising chromium(III) or a pharmaceutically acceptable salt thereof.

10. The composition of claim 9, wherein said chromium(III) or pharmaceutically acceptable salt thereof is present in an amount from about 50 μg to about 500 μg.

11. The composition of claim 2, further comprising a compound selected from the group consisting of β-carotene, lutein, zeaxanthin, lycopene, astaxanthin, flavonoids, resveratrol, phenolic compounds, anthocyanosides, essential fatty acids, and combinations thereof.

12. The composition of claim 3, further comprising a compound selected from the group consisting of β-carotene, lutein, zeaxanthine, lycopene, astaxanthin, flavonoids, resveratrol, phenolic compounds, anthocyanosides, essential fatty acids, and combinations thereof.

13. The composition of claim 12, wherein the phenolic compounds comprise oligomeric proanthocyanidins, and the essential fatty acids comprise omega-3.

14. The composition of claim 9, further comprising a compound selected from the group consisting of β-carotene, lutein, zeaxanthine, lycopene, astaxanthin, flavonoids, resveratrol, phenolic compounds, anthocyanosides, essential fatty acids, and combinations thereof.

15. A nutritional or dietary composition comprising, on a daily dosing basis: (a) about 300-450 mg α-lipoic acid or a pharmaceutical equivalent thereof; (b) about 50-70 mg genistein or pharmaceutical equivalent thereof; (c) about 200-300 mg ascorbic acid or a pharmaceutical equivalent thereof; (d) about 200-350 mg α-tocopheryl acetate or a pharmaceutical equivalent thereof; (e) about 1.5-3.5 mg thiamine mononitrate or a pharmaceutical equivalent thereof; (f) about 15-30 mg niacinamide or a pharmaceutical equivalent thereof; and (g) about 2-4 mg pyridoxine hydrochloride or a pharmaceutical equivalent thereof.

16. A nutritional or dietary composition comprising, on a daily dosing basis: (a) about 300-450 mg α-lipoic acid or a pharmaceutical equivalent thereof; (b) about 50-70 mg genistein or pharmaceutical equivalent thereof; (c) about 200-300 mg ascorbic acid or a pharmaceutical equivalent thereof; (d) about 200-350 mg α-tocopheryl acetate or a pharmaceutical equivalent thereof; (e) about 1.5-3.5 mg thiamine mononitrate or a pharmaceutical equivalent thereof; (f) about 15-30 mg niacinamide or a pharmaceutical equivalent thereof; (g) about 2-4 mg pyridoxine hydrochloride or a pharmaceutical equivalent thereof; and (h) about 100-250 μg chromium(III) chloride hexahydrate.

17. A nutritional or dietary composition comprising, on a daily dosing basis: (a) about 300-450 mg α-lipoic acid or a pharmaceutical equivalent thereof; (b) about 50-70 mg genistein or pharmaceutical equivalent thereof; (c) about 200-300 mg ascorbic acid or a pharmaceutical equivalent thereof; (d) about 200-350 mg α-tocopheryl acetate or a pharmaceutical equivalent thereof; (e) about 1.5-3.5 mg thiamine mononitrate or a pharmaceutical equivalent thereof, (f) about 15-30 mg niacinamide or a pharmaceutical equivalent thereof; (g) about 24 mg pyridoxine hydrochloride or a pharmaceutical equivalent thereof, and (h) about 100-250 μg chromium(III) picolinate.

18. A method for maintaining, strengthening, improving, or promoting ocular health of a subject, the method comprising administering to said subject a nutritional or dietary composition comprising, on a daily dosing basis: (a) α-lipoic acid or a pharmaceutical equivalent thereof; (b) ascorbic acid or a pharmaceutical equivalent thereof; and (c) α-tocopherol or a pharmaceutical equivalent thereof.

19. The method of claim 18, wherein the composition further comprises genistein or a pharmaceutical equivalent thereof.

20. The method of claim 18, wherein α-lipoic acid or a pharmaceutical equivalent thereof is present in an amount of about 50-1000 mg, ascorbic acid or a pharmaceutical equivalent thereof is present in an amount of about 50-1000 mg, and α-tocopherol or a pharmaceutical equivalent thereof is present in an amount of about 10-1000 mg.

21. The method of claim 20, wherein the composition further comprises genistein or a pharmaceutical equivalent thereof in an amount of about 20-200 mg.

22. The method of claim 20, wherein the subject suffers from, or is at risk of developing, an ocular disease.

23. The method of claim 22, wherein said ocular disease is selected from the group consisting of diabetic ocular complications, cataract, glaucoma, ocular inflammation, and combinations thereof.

24. The method of claim 22, wherein said ocular disease is diabetic retinopathy or diabetic macular edema.

25. The method of claim 21, wherein the subject suffers from, or is at risk of developing, an ocular disease.

26. The method of claim 25, wherein said ocular disease is selected from the group consisting of diabetic ocular complications, cataract, glaucoma, ocular inflammation, and combinations thereof.

27. The method of claim 25, wherein said ocular disease is diabetic retinopathy or diabetic macular edema.

28. The method of claim 18, wherein α-lipoic acid or a pharmaceutical equivalent thereof is present in an amount of about 100-600 mg, ascorbic acid or a pharmaceutical equivalent thereof is present in an amount of about 100-600 mg, and α-tocopherol or a pharmaceutical equivalent thereof is present in an amount of about 10-600 mg.

29. The method of claim 28, wherein the composition further comprises genistein or a pharmaceutical equivalent thereof in an amount of about 20-100 mg.

30. The method of claim 28, wherein the composition further comprises a material selected from the group consisting of vitamin B₁, vitamin B₃, vitamin B₆, pharmaceutically equivalents thereof, and combinations thereof.

31. The method of claim 30, wherein said material is present in an amount from about 100% to about 300% of an RDA thereof.

32. The method of claim 30, wherein said material is present in an amount from about 100% to about 200% of an RDA thereof.

33. The method of claim 31, wherein said composition further comprises chromium(III) or a pharmaceutically acceptable salt thereof.

34. The method of claim 33, wherein said chromium(III) or pharmaceutically acceptable salt thereof is present in an amount from about 50 μg to about 500 μg.

35. The method of claim 20, wherein said composition further comprises a compound selected from the group consisting of β-carotene, lutein, zeaxanthine, lycopene, astaxanthin, flavonoids, resveratrol, phenolic compounds, anthocyanosides, essential fatty acids, and combinations thereof.

36. The method of claim 21, wherein said composition further comprises a compound selected from the group consisting of β-carotene, lutein, zeaxanthine, lycopene, astaxanthin, flavonoids, resveratrol, phenolic compounds, anthocyanosides, essential fatty acids, and combinations thereof.

37. A method for maintaining, strengthening, improving, or promoting ocular health of a subject, the method comprising administering to said subject a nutritional or dietary composition comprising, on a daily dosage basis: (a) about 300-450 mg α-lipoic acid or a pharmaceutical equivalent thereof; (b) about 50-70 mg genistein or pharmaceutical equivalent thereof; (c) about 200-300 mg ascorbic acid or a pharmaceutical equivalent thereof; (d) about 200-350 mg α-tocopheryl acetate or a pharmaceutical equivalent thereof; (e) about 1.5-3.5 mg thiamine mononitrate or a pharmaceutical equivalent thereof; (f) about 15-30 mg niacinamide or a pharmaceutical equivalent thereof; and (g) about 2-4 mg pyridoxine hydrochloride or a pharmaceutical thereof.

38. A method for maintaining, strengthening, improving, or promoting ocular health of a subject, the method comprising administering to said subject a nutritional or dietary composition comprising, on a daily dosage basis: (a) about 300-450 mg α-lipoic acid or a pharmaceutical equivalent thereof; (b) about 50-70 mg genistein or pharmaceutical equivalent thereof; (c) about 200-300 mg ascorbic acid or a pharmaceutical equivalent thereof; (d) about 200-350 mg α-tocopheryl acetate or a pharmaceutical equivalent thereof; (e) about 1.5-3.5 mg thiamine mononitrate or a pharmaceutical equivalent thereof; (f) about 15-30 mg niacinamide or a pharmaceutical equivalent thereof; (g) about 2-4 mg pyridoxine hydrochloride or a pharmaceutical equivalent thereof; and (h) about 100-250 μg chromium(III) chloride hexahydrate or a pharmaceutical equivalent thereof.

39. A method for maintaining, strengthening, improving, or promoting ocular health of a subject, the method comprising administering to said subject a nutritional or dietary composition comprising, on a daily dosage basis: (a) about 300-450 mg α-lipoic acid or a pharmaceutical equivalent thereof; (b) about 50-70 mg genistein or pharmaceutical equivalent thereof; (c) about 200-300 mg ascorbic acid or a pharmaceutical equivalent thereof; (d) about 200-350 mg α-tocopheryl acetate or a pharmaceutical equivalent thereof; (e) about 1.5-3.5 mg thiamine mononitrate or a pharmaceutical equivalent thereof; (f) about 20-24 mg niacinamide; (g) about 2-4 mg pyridoxine hydrochloride or a pharmaceutical equivalent thereof; and (h) about 100-250 μg chromium(III) picolinate or a pharmaceutical equivalent thereof.

40. A method for producing a composition, the method comprising:
(a) providing amounts of ingredients which comprise: (1) α-lipoic acid or a pharmaceutical equivalent thereof, (2) ascorbic acid or a pharmaceutical equivalent thereof, and (3) α-tocopherol or a pharmaceutical equivalent thereof; and
(b) combining said amounts of said ingredients to produce said composition.

41. The method of claim 40, further comprising a step of forming said composition into a tablet, caplet, gel, capsule, solution, syrup, dispersion, emulsion, or patch.

42. The method of claim 40, further comprising providing an amount of genistein before or during the step of combining the ingredients.

43. The method of claim 40, further comprising providing an amount of a pharmaceutically acceptable carrier, as another ingredient, before the step of combining.

44. The method of claim 42, further comprising a step of forming said composition into a tablet, caplet, gel, capsule, solution, syrup, dispersion, emulsion, or patch.

45. The method of claim 40, wherein said α-lipoic acid or pharmaceutical equivalent thereof is present in an amount of about 50-1000 mg, said ascorbic acid or pharmaceutical equivalent thereof is present in an amount of about 50-1000 mg, and said α-tocopherol or pharmaceutical equivalent thereof is present in an amount of about 10-1000 mg.

46. The method of claim 42, wherein said genistein or pharmaceutical equivalent thereof is present in an amount of about 20-200 mg,
